(19) United States
(12) Patent Application Publication
(10) Pub. No.: US 2012/0277591 A1
(43) Pub. Date: Nov. 1, 2012

(54) ULTRASOUND DIAGNOSTIC APPARATUS

(76) Inventor: Hiroshi MURAKAMI, Kanagawa (JP)

(21) Appl. No.: 13/456,403

(22) Filed: Apr. 26, 2012

(30) Foreign Application Priority Data
Apr. 27, 2011 (JP) 2011-099428

(51) Int. Cl. A61B 8/14

(52) U.S. Cl. 600/445

(57) ABSTRACT

An ultrasound diagnostic apparatus capable of obtaining a high-quality ultrasound image while suppressing the temperature rise in an ultrasound probe. The ultrasound probe includes receive amplification units including amplifiers for amplifying reception signals outputted from a transducer array and a power supply unit for supplying bias current to the amplifiers. The power supply unit changes the bias current it supplies to the amplifiers according to the depth of a reflection position of ultrasonic echo.

### BACKGROUND OF THE INVENTION

The present invention relates to an ultrasound diagnostic apparatus and particularly to reduction of the amount of heat generated in an ultrasound probe of an ultrasound diagnostic apparatus for giving a diagnosis based on an ultrasound image produced by transmission and reception of ultrasonic waves to and from a transducer array of the ultrasound probe.

Conventionally, ultrasound diagnostic apparatus using ultrasound images are employed in medicine. In general, this type of ultrasound diagnostic apparatus comprises an ultrasound probe having a built-in transducer array and an apparatus body connected to the ultrasound probe. The ultrasound probe transmits ultrasonic waves toward a subject, receives ultrasonic echoes from the subject, and the apparatus body electrically processes the reception signals to generate an ultrasound image.

With such ultrasound diagnostic apparatus, heat is generated in the transducer array as the transducer array transmits ultrasonic waves.

The ultrasound probe is often encased in a housing of a size that can be readily held by an operator in a single hand because a diagnosis is normally given because the operator places the ultrasound transmission/reception surface of the transducer array in contact with a subject's surface by holding the ultrasound probe in a single hand. Therefore, the heat generated in the transducer array may raise the temperature inside the housing of the ultrasound probe.

In recent years, there has been proposed an ultrasound diagnostic apparatus having an ultrasound probe with a built-in circuit board for signal process including digital process of reception signals outputted from the transducer array before transmitting the reception signals to the apparatus body via wireless or wired communication in order to reduce the effects of noise and obtain a high-quality ultrasound image.

The ultrasound probe with digital processing is subject to generation of heat in the circuit board during processing of the reception signals, and therefore the temperature rise in the housing needs to be suppressed to assure stable operations of the circuits on the board.

As for a countermeasure against the temperature rise in the ultrasound probe, reference is made to, for example, JP 2005-253776 A describing an ultrasound diagnostic apparatus wherein the conditions for actuating the transducer array are automatically changed according to the surface temperature of the ultrasound probe. The surface temperature of the ultrasound probe is kept at an appropriate temperature by reducing, for example, driving voltage, number of transmission apertures, repetition frequency of the transmission pulse, and the frame rate as the surface temperature increases.

JP 2009-148424 A describes an ultrasound diagnostic apparatus where the operation of the reception circuit in the probe is halted for a given period of time such as freeze period, blanking period, period when the movement of the probe is within a specified value, and period when the temperature of the probe is above a specified value, in order to prevent temperature rise of the probe that may be caused by heat generated in circuits.

### SUMMARY OF THE INVENTION

However, the apparatus described in JP 2005-253776 A where the conditions for actuating the transducer array for transmission are changed cannot cope with the heat generated by the reception process in the ultrasound probe performing the above digital processing.

The apparatus described in JP 2009-148424 A where the operation of the reception circuit in the probe is halted for a given period of time such as freeze period, blanking period, period when the movement of the probe is within a specified value, and period when the temperature of the probe is above a specified value, may be capable of reducing the heat generated during reception in the ultrasound probe performing digital processing but is incapable of sufficiently reducing the heat in the ultrasound probe because the ratio of these specified periods to the operation time is considerably small.

An object of the present invention is to eliminate the above problems associated with the prior art and provide an ultrasound diagnostic apparatus enabling acquisition of a high-quality ultrasound image while suppressing the temperature rise inside the ultrasound probe.

To achieve the above object, the present invention provides an ultrasound diagnostic apparatus comprising an ultrasound probe including a transducer array for transmitting ultrasonic waves and receiving ultrasonic echo reflected by a subject to output reception signals corresponding to received ultrasonic waves, signal processors each including a receive amplification unit having an amplifier for amplifying reception signals outputted by the transducer array to process the reception signals, and a power supply unit for supplying bias current to the amplifier and a diagnostic apparatus body for producing an ultrasound image corresponding to the reception signals processed by the signal processors of the ultrasound probe, wherein the power supply unit changes a current value of the bias current supplied to the amplifier according to a depth of a reflection position of the ultrasonic echo.

Preferably, the power supply unit increases the bias current supplied to the amplifier as the depth of a reflection position of the ultrasonic echo increases.

Preferably, the receive amplification unit includes a plurality of amplifiers and amplifies the received signals through multiple stages, and the power supply unit variables a current value of a bias current at first-stage amplifier according to the depth of a reflection position of the ultrasonic echo.

Preferably, the receive amplification unit includes a low-noise amplifier, and wherein the power supply unit changes a current value of a bias current supplied to the low-noise amplifier according to the depth of a reflection position of the ultrasonic echo.

Preferably, the ultrasound probe includes an analog/digital converter for converting the reception signals into digital signals, and the analog/digital converter changes a sampling rate used in converting the received signals into digital signals according to the depth of a reflection position of the ultrasonic echo.

Preferably, the analog/digital converter changes the sampling rate used in converting the received signals into digital signals according to time elapsed as from transmission of the ultrasonic wave.

Preferably, the analog/digital converter lowers the sampling rate used in converting the received signals into digital signals as the depth of a reflection position of the ultrasonic echo increases.

Preferably, the ultrasound probe transmits and receives the reception signals to and from the diagnostic apparatus body via wireless communication.

Preferably, the power supply unit changes the current value of a bias current supplied to the amplifier and/or the low-noise amplifier according to the time elapsed as from transmission of the ultrasonic wave.

According to the present invention, the ultrasound probe comprises a receive amplification unit including an amplifier for amplifying reception signals outputted from a transducer array and a power supply unit for supplying bias current to the amplifier. The power supply unit changes the bias current it supplies to the amplifier according to the depth of the reflection position of an ultrasonic echo, achieving acquisition of a high-quality ultrasound image while saving on power consumption of the ultrasonic probe and thereby reducing the amount of heat generated in the ultrasound probe.

### BRIEF DESCRIPTION OF THE DRAWINGS

FIG. 1 is a block diagram illustrating a configuration of an ultrasound probe of the ultrasound diagnostic apparatus of the invention.

FIG. 2 is a block diagram illustrating a configuration of a diagnostic apparatus body of the ultrasound diagnostic apparatus of the invention.

FIG. 3 illustrates the concept of a transmission period and a reception period of ultrasonic waves.

FIG. 4A is a graph illustrating the concept of relation between depth and the bias current of a low-noise amplifier (LNA); FIG. 4B is a graph illustrating the concept of relation between depth and sampling rate.

### DETAILED DESCRIPTION OF THE INVENTION

Next, the ultrasound diagnostic apparatus of the invention is described in detail below with reference to the preferred embodiments shown in the accompanying drawings.

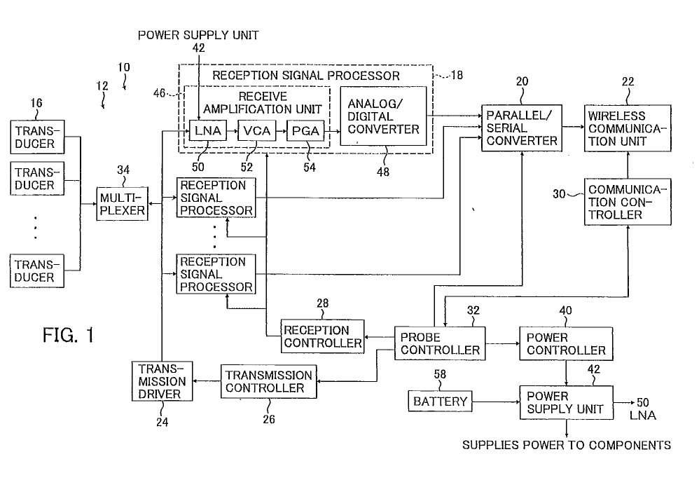

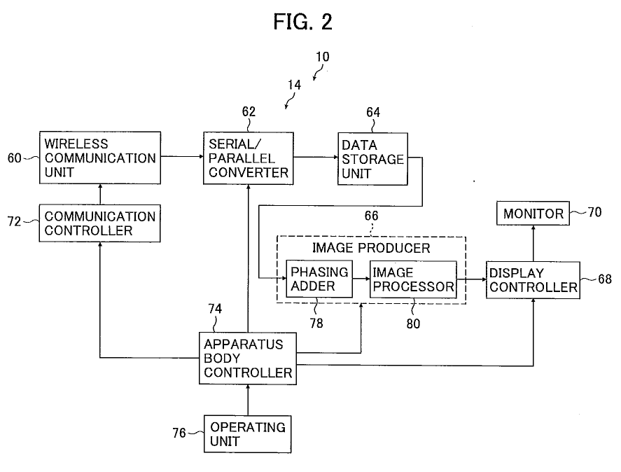

FIG. 1 is a block diagram illustrating the concept of a configuration of an ultrasound probe of the ultrasound diagnostic apparatus of the invention; FIG. 2 is a block diagram illustrating the concept of a configuration of a diagnostic apparatus body of the ultrasound diagnostic apparatus of the invention.

An ultrasound diagnostic apparatus 10 comprises an ultrasound probe 12 and a diagnostic apparatus body 14 that is connected to the ultrasound probe 12 via wireless communication.

The ultrasound probe 12 comprises a plurality of ultrasound transducers 16 constituting a plurality of channels of a unidimensional or two-dimensional transducer array, and the transducers 16 are connected via a multiplexer 34 to respective reception signal processors 18, which in turn are connected to a wireless communication unit 22 via a parallel/serial converter 20. The transducers 16 are connected to a transmission controller 26 via a transmission driver 24, and the reception signal processors 18 are connected to a reception controller 28, while the wireless communication unit 22 is connected to a communication controller 30.

The ultrasound probe 12 includes a power supply unit 42 for supplying electricity to components in the ultrasound probe 12; the power supply unit 42 is connected to a power controller 40 and a battery 58. The parallel/serial converter 20, the transmission controller 26, the reception controller 28, the communication controller 30, and the power controller 40 are connected to a probe controller 32.

The transducers 16 each transmit ultrasonic waves according to driving signals supplied from the transmission driver 24 and receive ultrasonic echoes from a subject to output reception signals. Each of the transducers 16 is composed of a vibrator comprising a piezoelectric body and electrodes each provided on both ends of the piezoelectric body. The piezoelectric body may be composed, for example, of a piezoelectric ceramic typified by a PZT (titanate zirconate lead), a polymeric piezoelectric device typified by PVDF (polyvinylidene flouride), or a piezoelectric monocrystal typified by PMN-PT (lead magnesium niobate lead titanate solid solution).

When the electrodes of each of the vibrators are supplied with a pulsed voltage or a continuous-wave voltage, the piezoelectric body expands and contracts to cause the vibrator to produce pulsed or continuous ultrasonic waves. These ultrasonic waves are combined to form an ultrasonic beam. Upon reception of propagating ultrasonic waves, each vibrator expands and contracts to produce an electric signal, which is then outputted as reception signal of the ultrasonic waves.

The multiplexer 34 selects an M number of ultrasound transducers from an N number of ultrasound transducers and connects the selected M number of ultrasound transducers to an M number of transmission and reception circuits respectively.

The transmission driver 24 includes, for example, a plurality of pulsers and adjusts the delay amounts of driving signals based on a transmission delay pattern selected by the transmission controller 26 so that the ultrasonic waves transmitted from the transducers 16 form a broad ultrasonic beam covering an area of a tissue inside the subject and supplies the transducers 16 with delay-adjusted driving signals via the multiplexer 34.

The shape of an ultrasonic beam transmitted from the transducers 16 is not limited to a wide shape; the beam may have a normal, narrowed-down shape.

The reception signal processor 18 of each channel processes the reception signal outputted from the corresponding transducer 16 under the control of the reception controller 28 and produces sample data containing area information on a tissue.

The reception signal processors 18 each comprise a receive amplification unit 46 and an analog/digital converter 48.

Each receive amplification unit 46 amplifies the received signal outputted from the corresponding transducer 16.

Each receive amplification unit 46 comprises an LNA (low noise amplifier) 50, a VCA (voltage controlled attenuator) 52, and a PGA (programmable gain amplifier) 54.

Each LNA 50 is supplied with bias current from the power supply unit 42 and amplifies the reception signal outputted from the corresponding transducer 16. Each LNA 50 acquires an increasingly greater S/N ratio as the supplied bias current increases.

According to the invention, the current value of the bias current supplied from the power supply unit 42 to each LNA is controlled by the power controller 40 described later so as to change according to the depth of the reflection position of ultrasonic echo, i.e., the time elapsed from the transmission of an ultrasonic beam.

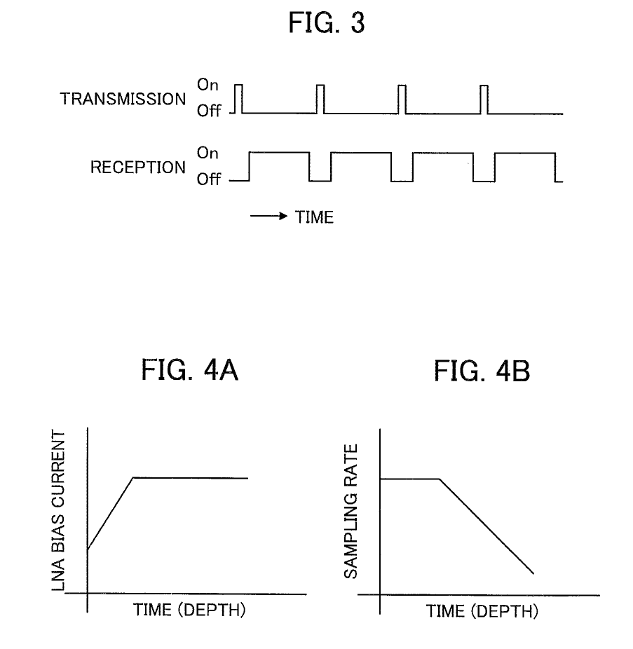

FIG. 3 illustrates the concept of a transmission period and a reception period of ultrasonic waves.

As illustrated in FIG. 3, the transducers 16 transmit an ultrasonic beam at given intervals for a given period of time according to the control by the transmission controller 26. In accordance with this, the reception signal processors 18, under the control by the reception controller 28, acquire the reception signals the transducers 16 output after having received ultrasonic echoes during a given period of time from transmission of an ultrasonic beam to transmission of the next ultrasonic beam.

As the ultrasonic echo is reflected at an increasingly deeper position of a subject, an increasingly longer period of time elapses from the transmission of an ultrasonic beam to the reception thereof. Accordingly, the current value of the bias current supplied to the LNA 50 is changed according to the ultrasonic echo acquisition time (the time elapsed from the transmission of ultrasonic waves)

FIG. 4A illustrates the concept of a relation between the value of the bias current supplied to the LNA 50 and the elapsed time (depth).

As illustrated in FIG. 4A, the value of the bias current supplied to the LNA 50 is so changed as to increase with the elapsed time counted from the transmission of ultrasonic waves, that is, with the depth of the reflection position of ultrasonic echo. The relation between the value of the bias current supplied to the LNA 50 and the elapsed time (depth) is predetermined according to, for example, the configuration of the apparatus.

An ultrasonic echo reflected at a deep position attenuates when passing through the subject and thus results in a weak reception signal in an ultrasound image. Therefore, the value of the bias current supplied to the LNA 50 for amplifying the reception signal is made increasingly greater as the ultrasonic echo is reflected at a deeper position in order to improve the S/N ratio and reduce the noise.

On the other hand, an ultrasonic echo reflected at a shallow position attenuates only to a small degree and hence yields strong reception signals, so that a high quality ultrasound image can be obtained with a low S/N ratio by reducing the current value of the bias current supplied to the LNA 50.

As described above, with a configuration whereby the operation of the reception circuit in the probe is halted for a given period of time such as freeze period, blanking period, period when the movement of the probe is within a specified value, and period when the temperature of the probe is above a specified value, reducing the heat of the ultrasound probe sufficiently is impossible because the ratio of these specified periods to the operation time is considerably small.

On the other hand, the ultrasound diagnostic apparatus of the invention achieves increase in S/N ratio and, hence, noise reduction by increasing the current value of the bias current supplied to the LNA 50 for amplifying the reception signal when the ultrasonic echo is reflected at a deep position, enabling acquisition of a high-quality ultrasound image even when the depth is great, and, when the depth of the ultrasonic echo is small, achieves saving on power consumed by the ultrasound probe 12 and hence suppression of temperature rise inside the ultrasound probe 12 by reducing the current value of the bias current supplied to the LNA 50.

The LNA 50 or the first stage of the receive amplification unit 46 for amplifying the reception signal outputted from the corresponding transducer 16 consumes power in a high ratio to the power consumption by the whole ultrasound probe 12. Therefore, more advantageous saving on power consumed by the ultrasound probe 12 is achieved and temperature rise inside the ultrasound probe 12 can be suppressed by reducing the current value of the bias current supplied to the first stage of the LNA 50 that consumes a great amount of power.

The LNA 50 supplies an amplified reception signal to the VCA 52.

In response to the instruction by a TGC (time gain control), not shown, the VCA 52 attenuates the reception signal supplied from the LNA 50 according to the depth of the reception signal. The VCA 52 supplies the amplified reception signal to the PGA 54.

The PGA 54 amplifies the reception signal supplied from the VCA 52 and supplies the amplified reception signal to the analog/digital converter 48.

The analog/digital converter 48 samples the analog reception signal supplied from the PGA 54 to produce digital sample data.

According to a preferred configuration of the invention, the analog/digital converter 48 changes the sampling rate used in sampling of the analog reception signal supplied from the PGA 54 according to the depth of the reflection position of an ultrasonic echo, i.e., the elapsed time counted from the transmission of an ultrasonic beam under the control by the reception controller 28.

FIG. 4B illustrates the concept of a relation between sampling rate used by the analog/digital converter 48 for sampling the reception signal and elapsed time counted from transmission of ultrasonic wave (depth).

As illustrated in FIG. 4B, the sampling rate used by the analog/digital converter 48 for sampling the reception signal is changed so as to decrease as the elapsed time counted from the transmission of ultrasonic wave increases, that is, as the depth of the reflection position of ultrasonic echo increases. The relation between sampling rate and elapsed time (depth) is predetermined according to, for example, the configuration of the apparatus.

Generally, the analog/digital converter 48 samples the reception signal at a high sampling rate of 40 MHz to 50 MHz.

As described above, an ultrasonic echo reflected at a deep position attenuates as it passes through the subject. The high frequency component, in particular, attenuates so greatly that the frequency of the ultrasonic echo is about several MHz. Therefore, when sampling a reception signal having a great depth, the analog/digital converter 48 is capable of correct sampling even with a lower sampling rate than a normal sampling rate. A low sampling rate of which the analog/digital converter 48 is capable permits reduction in power consumed by the analog/digital converter 48 and hence saving on power consumption as well as suppression of temperature rise inside the ultrasound probe 12.

Like the LNA 50 described above, the analog/digital converter 48 consumes power in a high ratio to the whole power consumption by the ultrasound probe 12. Thus, by lowering the sampling rate used by the analog/digital converter 48 for sampling the reception signal, more advantageous saving on power consumed by the ultrasound probe 12 is achieved and temperature rise inside the ultrasound probe 12 can be suppressed.

The analog/digital converter 48 supplies the sample data to the parallel/serial converter 20.

The parallel/serial converter 20 converts the parallel sample data generated by a plurality of channels of the reception signal processors 18 into serial sample data.

The wireless communication unit 22 performs carrier modulation based on serial sample data to generate transmission signals and supplies an antenna with the transmission signals so that the antenna transmits radio waves to transmit serial sample data. The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 22 transmits the sample data to the diagnostic apparatus body 14 through wireless communication with the diagnostic apparatus body 14, receives various control signals from the diagnostic apparatus body 14, and outputs the received control signals to the communication controller 30. The communication controller 30 controls the wireless communication unit 22 so that the sample data are transmitted with a transmission wave intensity that is set by the probe controller 32 and outputs various control signals received by the wireless communication unit 22 to the probe controller 32.

The probe controller 32 controls components of the ultrasound probe 12 according to various control signals transmitted from the diagnostic apparatus body 14.

The ultrasound probe 12 may be an external type probe such as linear scan type, convex scan type, and sector scan type or a probe of, for example, a radial scan type used for an ultrasound endoscope.

The power supply unit 42 supplies the charged power in the battery 58 to components of the ultrasound probe 12 such as the transmission driver 24 and reception signal processors 18 under the control by the power controller 40.

In response to an instruction from the probe controller 32, the power controller 40 controls the power supply unit 42 to supply given amounts of electricity to components of the ultrasound probe 12.

According to an instruction signal from the probe controller 32, the power controller 40 controls the power supply unit 42 to change the current value of the bias current supplied to the LNA 50 of each reception signal processor 18.

On the other hand, the diagnostic apparatus body 14 comprises a wireless communication unit 60, which is connected to a data storage unit 64 via a serial/parallel converter 62. The data storage unit 64 is connected to an image producer 66. The image producer 66 is connected to a monitor 70 via a display controller 68.

The wireless communication unit 60 is also connected to a communication controller 72; the serial/parallel converter 62, the image producer 66, the display controller 68, and the communication controller 72 are connected to an apparatus body controller 74. In addition, the apparatus body controller 74 is connected to an operating unit 76 for an operator to perform input operations.

The operating unit 76 is provided to set an imaging menus and imaging conditions and to input instructions for imaging of a subject. The operating unit 76 is provided for the operator to perform input operations and may be composed of, for example, a keyboard, a mouse, a track ball, and/or a touch panel.

The wireless communication unit 60 transmits various control signals to the ultrasound probe 12 through wireless communication with the ultrasound probe 12. The wireless communication unit 60 demodulates the signal received by the antenna to output serial sample data.

The communication controller 72 controls the wireless communication unit 60 so that various control signals are transmitted with a transmission radio wave intensity that is set by the apparatus body controller 74.

The serial/parallel converter 62 converts the serial sample data outputted from the wireless communication unit 60 into parallel sample data. The data storage unit 64 is configured by, for example, a memory or a hard disk and stores at least one frame of sample data converted by the serial/parallel converter 62.

The image producer 66 performs reception focusing on each frame of sample data read out from the data storage unit 64 to generate an image signal representing an ultrasound diagnostic image. The image producer 66 includes a phasing adder 78 and an image processor 80.

The phasing adder 78 selects one reception delay pattern from a plurality of previously stored reception delay patterns according to the reception direction set by the apparatus body controller 74 and, based on the selected reception delay pattern, provides complex baseband signals represented by the sample data with respective delays and adds them up in reception focusing processing. This reception focusing yields a baseband signal or a sound ray signal where the ultrasonic echoes are well focused.

The image processor 80 generates a B-mode image signal, which is tomographic image information on a tissue inside the subject, according to the sound ray signal generated by the phasing adder 78. The image processor 80 includes an STC (sensitivity time control) section and a DSC (digital scan converter). The STC section corrects the sound ray signal for the attenuation due to distance according to the depth of the reflection position of the ultrasonic waves. The DSC converts the sound ray signal corrected by the STC into an image signal compatible with the scanning method of an ordinary television signal through raster conversion and generates a B mode image signal through required image processing such as contrast processing.

The display controller 68 causes the monitor 70 to display an ultrasound diagnostic image according to the image signals generated by the image producer 66. The monitor 70 includes a display device such as an LCD, for example, and displays an ultrasound diagnostic image under the control by the display controller 68.

The apparatus body controller 74 controls components of the ultrasound diagnostic apparatus 10 according to the operations performed by the operator using the operating unit 76.

While the serial/parallel converter 62, the image producer 66, the display controller 68, the communication controller 72, and the apparatus body controller 74 in the diagnostic apparatus body 14 are each constituted by a CPU and an operation program for causing the CPU to perform various kinds of processing, they may each be constituted by a digital circuit.

Next, the operation of the ultrasound diagnostic apparatus 10 is described.

When the operator brings the ultrasound probe 12 into contact with the surface of a subject and starts imaging, the transmission controller 26 controls the transmission driver 24 according to a control signal from the apparatus body controller 74. The transmission driver 24 drives the transducers 16 according to the control signal, and the transducers 16 transmit an ultrasonic beam and receive ultrasonic echo from the subject to produce reception signals.

The reception signals outputted from the transducers having received the ultrasonic echo from the subject are respectively supplied to the reception signal processors 18. The reception signals supplied to the reception signal processors 18 are sequentially converted into sample data and serialized through the parallel/serial converter 20 before being wirelessly transmitted from the wireless communication unit 22 to the diagnostic apparatus body 14. The sample data received by the wireless communication unit 60 of the diagnostic apparatus body 14 are converted into parallel data through the serial/parallel converter 62 and stored in the data storage unit 64. Further, the sample data are read out from the data storage unit 64 frame by frame, and the image producer 66 generates image signals, based on which image signals the display controller 68 causes the monitor 70 to display an ultrasound diagnostic image.

According to the invention, the current value of the bias current supplied to the LNA 50 is changed depending on the depth of the ultrasonic echo. Specifically, when an ultrasonic echo reflected (reception signal) from a shallow position and hence having a great signal strength is amplified, the current value of the bias current supplied to the LNA 50 is reduced. On the other hand, when an ultrasonic echo reflected (reception signal) from a deep position is to be amplified, the current value of the bias current supplied to the LNA 50 is increased.

Since the current value of the bias current supplied to the LNA 50 is changed according to the depth of the ultrasonic echo, a high-quality ultrasound image can be obtained even when the depth is great, and when the depth is small, the current value of the bias current supplied to the LNA 50 is reduced to achieve saving on power consumed by the ultrasound probe 12 and, hence, suppression of temperature rise inside the ultrasound probe 12 that performs digital processing.

Further, in a preferred mode, since the analog/digital converter 48 changes the sampling rate at which it samples the reception signals according to the depth of the ultrasonic echo, the reception signals corresponding to a great depth and, hence, not requiring a high sampling rate can be sampled at a low sampling rate, saving on power consumed by the analog/digital converter 48.

The present invention is basically as described above.

While the present invention has been described in detail above, the invention is not limited to the above embodiments and various modifications and improvements may be made without departing from the spirit of the invention.

For example, the receive amplification unit 46 for amplifying the reception signals from the transducers includes LNA 50, VCA 52, and PGA 54 in the illustrated ultrasound diagnostic apparatus but may, without limitation thereto, additionally comprise other amplifiers or may be composed of other amplifiers.

Further, in the illustrated example, the bias current supplied to the LNA 50 is switched but the invention is not limited thereto; the bias current supplied to the PGA 54 may be switched, or the bias currents supplied to the LNA 50 and the PGA 54 may be both switched. Where there are provided a plurality of amplifiers for amplifying the reception signals from the transducers, the bias current supplied to at least one of these amplifiers need only be switched.

In the illustrated example, the bias current to the LNA 50 and the sampling rate used by the analog/digital converter 48 are adapted to linearly change in proportion to depth as illustrated in FIGS. 4A and 4B but, without limitation thereto, may be switched to change in a curved line or in a step-wise manner.

Further, in the illustrated example, the ultrasound probe 12 and the diagnostic apparatus body 14 exchange signals via wireless communication but, without limitation thereto, may exchange signals via wired communication.

We claim:

1. An ultrasound diagnostic apparatus comprising
an ultrasound probe including
a transducer array for transmitting ultrasonic waves and receiving ultrasonic echo reflected by a subject to output reception signals corresponding to received ultrasonic waves,
signal processors each including a receive amplification unit having an amplifier for amplifying reception signals outputted by the transducer array to process the reception signals; and
a power supply unit for supplying bias current to the amplifier and
a diagnostic apparatus body for producing an ultrasound image corresponding to the reception signals processed by the signal processors of the ultrasound probe,
wherein the power supply unit changes a current value of the bias current supplied to the amplifier according to a depth of a reflection position of the ultrasonic echo.

2. The ultrasound diagnostic apparatus according to claim 1, wherein the power supply unit increases the bias current supplied to the amplifier as the depth of a reflection position of the ultrasonic echo increases.

3. The ultrasound diagnostic apparatus according to claim 1, wherein the receive amplification unit includes a plurality of amplifiers and amplifies the reception signals through multiple stages, and wherein the power supply unit changes a current value of a bias current supplied to a first-stage amplifier according to the depth of a reflection position of the ultrasonic echo.

4. The ultrasound diagnostic apparatus according to claim 1, wherein the receive amplification unit includes a low-noise amplifier, and wherein the power supply unit changes a current value of a bias current supplied to the low-noise amplifier according to the depth of a reflection position of the ultrasonic echo.

5. The ultrasound diagnostic apparatus according to claim 1,
wherein the ultrasound probe includes an analog/digital converter for converting the reception signals into digital signals, and
wherein the analog/digital converter changes a sampling rate used in converting the reception signals into digital signals according to the depth of a reflection position of the ultrasonic echo.

6. The ultrasound diagnostic apparatus according to claim 5, wherein the analog/digital converter lowers the sampling rate used in converting the reception signals into digital signals as the depth of a reflection position of the ultrasonic echo increases.

7. The ultrasound diagnostic apparatus according to claim 6, wherein the analog/digital converter changes the sampling rate used in converting the reception signals into digital signals according to time elapsed as from transmission of the ultrasonic wave.

8. The ultrasound diagnostic apparatus according to claim 1, wherein the ultrasound probe transmits and receives the reception signals to and from the diagnostic apparatus body via wireless communication.

9. The ultrasound diagnostic apparatus according to claim 1, wherein the power supply unit changes the current value of a bias current supplied to the amplifier and/or the low-noise amplifier according to the time elapsed as from transmission of the ultrasonic wave.
